(19) United States
(12) Patent Application Publication
(10) Pub. No.: US 2013/0060174 A1
(43) Pub. Date: Mar. 7, 2013

(54) ASSEMBLY HAVING A BODY OF ABSORBENT MATERIAL AND A CAPSULE INSERTED THEREIN

(71) Applicants: Kilian Rolli, Baden (CH); David Conroy, New York, NY (US)

(72) Inventors: Kilian Rolli, Baden (CH); David Conroy, New York, NY (US)

(21) Appl. No.: 13/696,722

(22) PCT Filed: May 23, 2011
(86) PCT No.: PCT/EP2011/058364

§ 371 Date: Nov. 7, 2012

(30) Foreign Application Priority Data
May 27, 2010 (EP) 10164008.4

(51) Int. Cl. A61F 13/20; A61H 1/00

(52) U.S. Cl. 601/46; 604/15

(57) ABSTRACT

Assembly having a body (2) of absorbent material and a capsule (3) inserted therein. The capsule (3) has at least one projection (6, 11) that is arranged on its circumference along a helical line. For the manufacture of the assembly, the capsule (3) is screwed into the body (2) by a combined rotary and translational movement.

The invention relates to an assembly having a body of absorbent material and a capsule inserted therein.

From references U.S. Pat. No. 5,782,779, U.S. Pat. No. 6,183,428, and U.S.2007/0260210, tampon assemblies having a vibration element embedded in the tampon body are known in the art. Tampon assemblies of this kind are used to relieve menstrual problems. As to the embedding of the vibration element in the tampon body, it follows from US2007/0260210 only that the vibration element is contained in a capsule on the exterior of which retaining elements are arranged in order to prevent that the capsule is pulled out of the tampon body particularly when the assembly is retracted from the vagina. In this reference, the tampon body is described as a hollow body into which the capsule is apparently inserted.

On the background of this prior art, the invention is based upon the object of providing an assembly that is simple to manufacture and where the capsule is firmly anchored in the body.

According to the invention, this object is attained in that the capsule has at least one projection that is arranged on its circumference along a helical line.

This solution of the invention offers the advantage that the at least one projection acts like a thread, thereby facilitating the introduction of the capsule and ensuring a firm anchorage of the capsule in the body.

According to one embodiment, the projection has the shape of a helical ridge. This allows a simple manufacture of the capsule.

According to another embodiment, the ridge is provided with incisions on its external circumference. These incisions increase the resistance against an involuntary backward rotation of the capsule.

If according to another embodiment the incisions each have two flanks that are oriented in the direction of the helical line and have different inclinations, the resistance against backward rotation can be increased without making the introduction more difficult.

According to an alternative embodiment, multiple projections are arranged along a helical line. In this manner, the anchorage of the capsule in the body is improved.

According to another embodiment, each projection has two flanks that are oriented in the direction of the helical line and have different inclinations. In this manner, the capsule is relatively easy to screw in but has a considerable resistance against backward rotation.

According to another embodiment, the circumferential surface of the capsule and/or the projection has tangentially extending protrusions whose free ends are oriented in the direction of the helical line and opposed to the screw introduction direction. These protrusions have the function of barbs and provide additional anchorage of the capsule in the body.

According to another embodiment, the body has an elongate shape. Such a shape is particularly suitable for a tampon for women's menstrual hygiene or for medical applications.

With regard to the design of the assembly as a tampon it is advantageous if according to another embodiment the absorbent material is a fibrous material.

If according to another embodiment the capsule has an elongate shape with an introduction end and a rear end, it is well suited for being received in a body having an elongate shape.

According to another embodiment, the capsule is cylindrical on at least part of its length. This shape provides a firm contact between the body and the capsule after its insertion.

With regard to the insertion of the capsule into the body it is advantageous if according to another embodiment the capsule has a pointed end.

According to another embodiment, the height of the projection or of the projections, respectively, increases from the introduction end of the capsule. This facilitates the insertion of the capsule into the body. If according to another embodiment the mentioned height decreases toward the rear end of the capsule, an involuntary separation of the capsule from the body becomes more difficult or even impossible, particularly if the body, as described above, is constricted after the introduction of the capsule in the region of the rear end of the capsule as seen in the introduction direction.

According to another embodiment, a flexible pulling means is fastened to the capsule. This pulling means is particularly intended for retracting an assembly that is being worn in an orifice of the body.

According to a particular embodiment, at least one conductor or conduit is arranged in the pulling means. The latter may be a conductor for electric current or a conduit for a fluid.

According to another embodiment, an energy consumer and/or a sensor is arranged inside the capsule. This enables various applications of the assembly, particularly in the field of medicine.

Finally, according to one embodiment, the energy consumer is a vibration generator. Such an assembly is suitable for the treatment of menstrual problems.

The invention further refers to a method for manufacturing an assembly of the previously described kind.

The object of the method is to introduce and firmly anchor a capsule in a body from fibrous material with little effort.

This object is attained in that the capsule is screwed into the body by a combined rotary and translational movement.

This solution of the invention offers the advantage that the capsule can be introduced into the body by a screwing motion with relatively little force expenditure.

According to one embodiment, prior to the introduction of the capsule, the body is provided with an opening for screwing in the capsule. In this manner, the force required for introducing the capsule is further reduced.

If according to another embodiment the body is constricted after the introduction of the capsule in the region of the rear end of the capsule as seen in the introduction direction, the safety against pulling out the capsule from the body increases. Advantageously, the constriction is achieved by the application of heat.

According to another embodiment, the capsule is fastened inside the body by a thread. This measure also serves for an improved anchorage of the capsule inside the body.

For a further improved anchorage of the capsule, a capsule with protrusions as described above may be used and turned back a certain amount after its introduction.

Exemplary embodiments of the invention will be explained by way of examples in more detail hereinafter with reference to the accompanying drawings showing

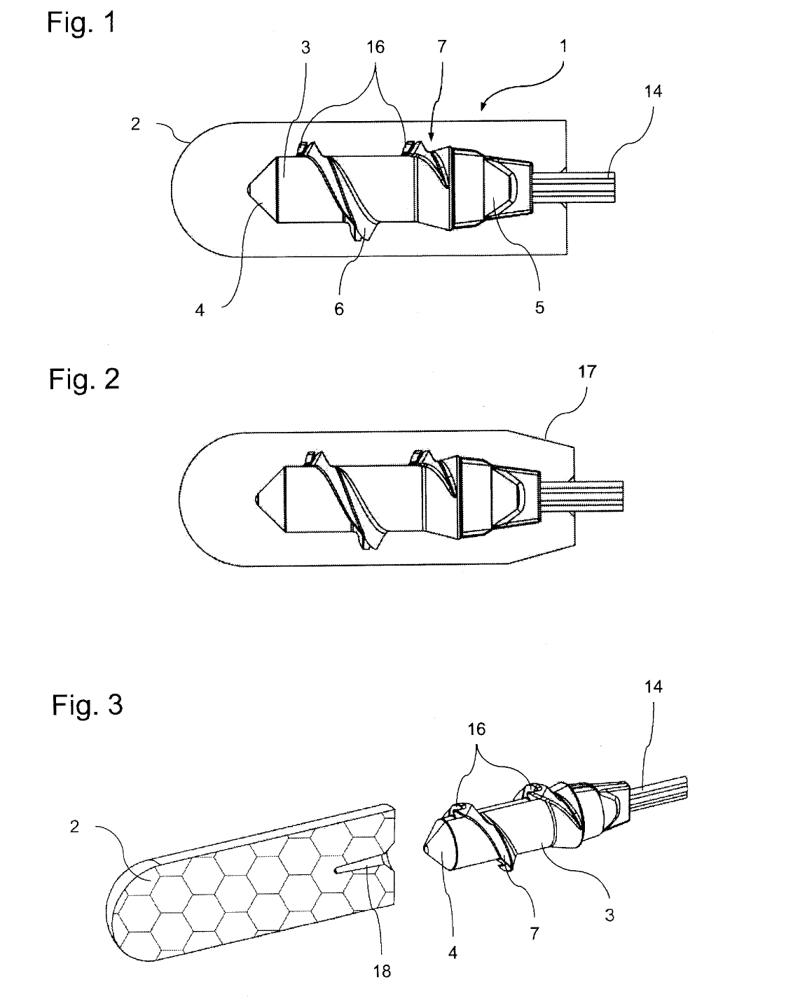

FIG. 1 a lateral view of an assembly of an absorbent body and a capsule,

FIG. 2 a view as in FIG. 1 after the constriction of the absorbent body,

FIG. 3 a perspective view of the absorbent body and the capsule prior to their assembly,

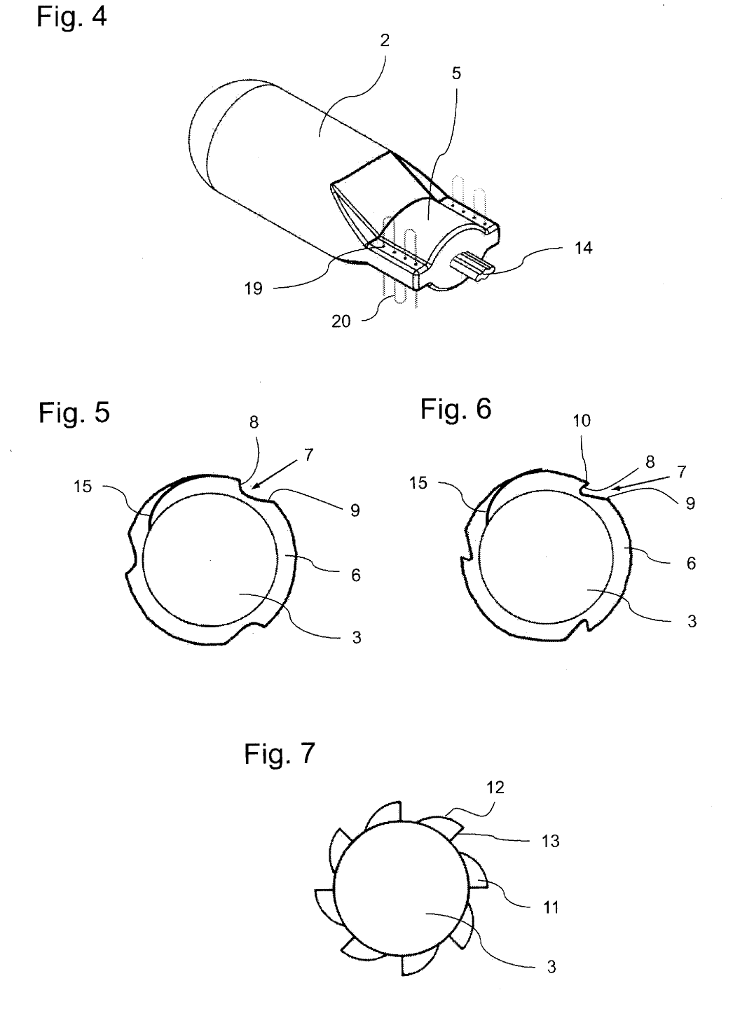

FIG. 4 a perspective view of another embodiment of the assembly,

FIG. 5 a front view of another embodiment of the capsule,

FIG. 6 a front view of yet another embodiment of the capsule, and

FIG. 7 a front view of yet another embodiment of the capsule.

FIG. 1 shows a lateral view of an assembly 1 of a body 2 and a capsule 3 arranged therein. The body consists of an absorbent material, e.g. a fibrous or an expanded material and is depicted by a contour line so that capsule 3 arranged therein is well visible. Assembly 1 may e.g. be a vibrating tampon as described in references U.S. Pat. No. 5,782,779, U.S. Pat. No. 6,183,428, and U.S.2007/0260210 cited in the introduction. However, the invention is not limited to vibrating tampons. Capsule 3 has a front end 4 which in the depicted example is pointed, and a rear end 5. On the circumference of capsule 3, a circumferential helical ridge 6 is arranged. In the assembly of capsule 3 and body 2, this ridge 6 acts like a single-start screw thread. Alternatively, two or more ridges 6 might be arranged on the circumference of capsule 3 that would correspondingly act like a double-start or a multiple screw thread. At the rear end 5 of capsule 3, a flexible pulling means is arranged, e.g. in the form of a cable 14. The higher the pitch of helical ridge 6, the higher the risk that capsule 3 will turn back out of body 2 under the action of a traction force applied to cable 14. To counteract this risk, capsule 3 is provided with barbs 16 whose points are directed against the screw insertion direction. Additionally, ridge 6 may be provided with incisions 7 whose design will be explained in more detail below with reference to FIG. 5.

As shown in FIG. 2, as an additional safety against an involuntary separation of capsule 3 from body 2 after the insertion of capsule 3, body 2 may be constricted in the area of rear end 5 of capsule 3. This constriction 17 may be achieved by applying a radial force in the mentioned area. In addition, to make this constriction permanent, heat may be applied during and/or after the deformation. If the body comprises synthetic fibers and/or a sheathing with synthetic fibers, these fibers may be partially melted by the heat and thus bond to adjacent fibers.

FIG. 3 shows a perspective view of body 2 that is cut open here, and of capsule 3 prior to its assembly with body 2. The rear end of the latter has previously been provided with an opening 18 in which capsule 3 is inserted by its front end 4 and subsequently screwed in. After fully screwing in capsule 3, the latter is preferably turned back a little so that barbs 16 arranged on its circumference penetrate into the fibrous material of body 2 and thus firmly anchor capsule 3 inside body 2. Subsequently, body 2 may be constricted in the area of rear end 5 of capsule 3 as described above.

FIG. 4 shows another measure for securing capsule inside body 2. For this purpose, holes 19 are provided in the rear end 5 of capsule 3 which allow attaching body 2 by means of a thread 20. It is also possible to constrict body 2 by means of a thread without guiding the thread through holes 19. For example, the thread may be guided through body 2 in the manner of a purse-string stitch as it is e.g. known from surgery.

FIG. 5 shows a schematic view of a capsule 3 as seen from its front end 4. The height of ridge 6 increases from zero to a maximum, this lead-in section being designated by the numeral 15 in the figure. Furthermore, the mentioned incisions 7 are clearly visible of which three are provided per turn of ridge 6 in the example. It is further apparent that each incision 7 has two flanks 8, 9, flank 8 being steeper than flank 9 in the depicted example. As a result, relatively flat flank 9 will cause a smaller resistance during the insertion of capsule 3 into body 2 than relatively steep flank 8 in the attempt to unscrew capsule 3 from body 2.

In the illustration of FIG. 6, the design of flanks 8, 9 of incisions 7 is chosen such that a point 10 similar to a sawtooth is formed at the outer end of flank 8. In the attempt to unscrew capsule 3 from body 2, points 10 will penetrate into the material of body 2 and thus prevent any further backward rotation.

FIG. 7 shows another example where instead of a continuous ridge 6 a plurality of projections 11 are arranged on the external circumference of capsule 3 along a helical line. Here also, similarly as in the examples that have been previously described with reference to FIGS. 5 and 6, flanks 12 and 13 of projections 11 may have different inclinations.

### List of Reference Numerals

1 assembly
2 body
3 capsule
4 front end
5 rear end
6 ridge
7 incision
8 flank of 7
9 flank of 7
10 point
11 projection
12 flank of 11
13 flank of 11
14 cable
15 lead-in
16 barb
17 constriction
18 opening
19 hole
20 thread

1. An assembly comprising:
a body of absorbent material; and
a capsule inserted therein, wherein the capsule has at least one projection that is arranged on its circumference along a helical line.

2. An assembly according to claim 1, wherein the projection has the shape of a helical ridge.

3. An assembly according to claim 2, wherein the ridge is provided with incisions on its external circumference.

4. An assembly according to claim 3, wherein the incisions each have two flanks that are oriented in the direction of the helical line and have different inclinations.

5. An assembly according to claim 1, wherein multiple projections are arranged along a helical line.

6. An assembly according to claim 5, wherein the projections each have two flanks that are oriented in the direction of the helical line and have different inclinations.

7. An assembly according to claim 1, wherein the circumferential surface of the capsule and/or the projection has tangentially extending protrusions whose free ends are oriented in the direction of the helical line and opposed to the screw introduction direction.

8. An assembly according to claim 1, wherein the body has an elongate shape.

9. An assembly according to claim 1, wherein the absorbent material is a fibrous material.

10. An assembly according to claim 1, wherein the capsule has an elongate shape with an introduction end and a rear end.

11. An assembly according to claim 1, wherein the capsule is cylindrical on at least part of its length.

12. An assembly according to claim 10, wherein the capsule is pointed at its introduction end.

13. An assembly according to claim 10, wherein the height of the projection or of the projections, respectively, increases from the introduction end of the capsule and preferably decreases toward the rear end of the capsule.

14. An assembly according to claim 1, wherein a flexible pulling means is fastened to the capsule.

15. An assembly according to claim 14, wherein at least one conductor or conduit is arranged in the pulling means.

16. An assembly according to claim 1, wherein an energy consumer and/or a sensor is arranged inside the capsule.

17. An assembly according to claim 16, wherein the energy consumer is a vibration generator.

18. A method for manufacturing an assembly according to claim 1, wherein the capsule is screwed into the body by a combined rotary and translational movement.

19. A method according to claim 18, wherein prior to the introduction of the capsule, the body is provided with an opening for screwing in the capsule is screwed.

20. A method according to claim 18, wherein the body is constricted after the introduction of the capsule in the region of the rear end of the capsule as seen in the introduction direction.

21. A method according to claim 20, wherein the constriction is achieved by the application of heat.

22. A method according to claim 18, wherein the capsule is fastened inside the body by a thread.

23. A method for manufacturing an assembly according to claim 7, wherein the capsule is screwed into the body by a combined rotary and translational movement and after introducing the capsule, the capsule is turned back a certain amount.
